(19) United States
(12) Patent Application Publication
(10) Pub. No.: US 2003/0148950 A1
(43) Pub. Date: Aug. 7, 2003

(54) Kringle domain 1 of human hepatocyte growth factor and uses therefor

(76) Inventors: Li Xin, Los Angeles, CA (US); Zai-Ping Li, Shanghai (CN); Ren-Bao Gan, Shanghai (CN); Qing-Wei Zhou, Shanghai (CN); Ren Xu, Albany, CA (US)

Correspondence Address: Peng Chen, Morrison & Foerster LLP, Suite 500, 3811 Valley Centre Drive, San Diego, CA, 92130-2332, US

(21) Appl. No.: 10/267,137

(22) Filed: Oct. 7, 2002

Related U.S. Application Data
(60) Provisional application No. 60/328,329, filed on Oct. 9, 2001.

(51) Int. Cl. A61K 38/18; C07H 21/04; C12P 21/02; C12N 5/06; C07K 14/475

(52) U.S. Cl. 514/12; 530/399; 536/23.5; 435/69.1; 435/320.1; 435/325

(57) ABSTRACT

This invention relates generally to the field of growth factor. In particular, the invention provides an isolated nucleic acid fragment, comprising a sequence of nucleotides encoding a Kringle domain 1 of human hepatocyte growth factor (HGFK1). Proteins or peptides encoded by the HGFK1 nucleic acids are also provided. Compositions comprising HGFK1 nucleic acids, proteins, or functional derivatives or fragments are also provided. Methods for producing and/or using HGFK1 nucleic acids, proteins, or functional derivatives or fragments are further provided.

### CROSS-REFERENCE TO RELATED APPLICATIONS

The present application claims priority benefit of the provisional U.S. Patent Application Serial No. 60/328,329, filed Oct. 9, 2001, the content of which is herein incorporated by reference in its entirety.

### FIELD OF INVENTION

This invention relates generally to the field of growth factor. In particular, the invention provides an isolated nucleic acid fragment, comprising a sequence of nucleotides encoding a Kringle domain 1 of human hepatocyte growth factor (HGFK1). Proteins or peptides encoded by the HGFK1 nucleic acids are also provided. Compositions comprising HGFK1 nucleic acids, proteins, or functional derivatives or fragments are also provided. Methods for producing and/or using HGFK1 nucleic acids, proteins, or functional derivatives or fragments are further provided.

### BACKGROUND OF THE INVENTION

Kringle domain is a kind of protein module, which usually consists of about 80 amino acids (1). There are two conservative clusters of amino acids and six conservative cysteines in it so as to form certain structure constrained by three pairs of inner disulfide bonds. Kringle domains were found in many proteins from one to about forty copies (2). For example, there are 2 copies in tissue plasminogen activator, 5 in plasminogen, 15 to 30 in apolipoprotein A. Previously, kringle modules in these proteins are thought to function as recognition units for binding of other proteins in solution and on cells (1).

Angiostatin is a circulating angiogenesis inhibitor that has been found to be part of plasminogen (3). It contains the first four kringles of plasminogen. The exact inhibitory mechanism is not completely clear yet. Each kringle of angiostatin and the five kringles of plasminogen were all cloned and proved to be angiogenesis inhibitors, with the fourth kringle to be exceptional (4). Kringle 5 is even more potent in inhibiting the growth of endothelial cell than angiostatin (5). Later, kringle 2 of prothrombin was found to be an angiogenesis inhibitor (6), too, while completely different results were reported as to whether apolipoprotein A has inhibitory effect on endothelial cell growth or not (7,8).

Hepatocyte growth factor (HGF), also known as scatter factor, is a mesenchymal or stromal-derived mediator with angiogenic activity (2). There are four kringles in its amino terminus, showing a remarkable sequence similarity with those of plasminogen. In order to see whether they exhibit anti-angiogenic activity as kringles of plasminogen or not, kringle 1 of HGF was cloned and expressed in E. coli. We demonstrated for the first time that kringle 1 of hepatocyte growth factor is a potent angiogenesis inhibitor and that treatment of BAE cells with kringle 1 of HGF caused cell apoptosis.

### BRIEF SUMMARY OF THE INVENTION

In one aspect, the invention provides an isolated nucleic acid fragment, comprising a sequence of nucleotides encoding a Kringle domain 1 of human hepatocyte growth factor (HGFK1). Preferably, the isolated HGFK1 nucleic acid fragment encodes the amino acid sequence set forth below:

(SEQ ID NO:1)

N′-CIIGKGRSYKGTVSITKSGIKCQPWSSMIPHEH-
SFLPSSYRGKDLQENYCRNPRGEEGGPWCFTSNPEVRYEVCDIPQC-C′
or

(SEQ ID NO:2)

N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

Also preferably, the isolated HGFK1 nucleic acid fragment is hybridizable, under low, middle or high stringency, with a nucleotide sequence set forth below (SEQ ID NO:3):

5′-
AACTGCATCA TTGGTAAAGG ACGCAGCTAC AAGGGAACAG
TATCTATCAC TTGACGTAGT AACCATTTCC TGCGTCGATG
TTCCCTTGTC ATAGATAGTG TAAGAGTGGC ATCAAATGTC
AGCCCTGGAG TTCCATGATA CCACACGAAC ATTCTCACCG
TAGTTTACAG TCGGGACCTC AAGGTACTAT GGTGTGCTTG
ACAGCTTTTT GCCTTCGAGC TATCGGGGTA AAGACCTACA
GGAAAACTAC TGTCGAAAAA CGGAAGCTCG ATAGCCCCAT
TTCTGGATGT CCTTTTGATG TGTCGAAATC CTCGAGGGGA
AGAAGGGGGA CCCTGGTGTT TCACAAGCAA ACAGCTTTAG
GAGCTCCCCT TCTTCCCCCT GGGACCACAA AGTGTTCGTT
TCCAGAGGTA CGCTACGAAG TCTGTGACAT TCCTCAGTGT
TCAGAAGTTG AGGTCTCCAT GCGATGCTTC AGACACTGTA
AGGAGTCACA AGTCTTCAAC AATGCATGAC CTGC-3′
TTACGTACTG GACG

More preferably, the isolated HGFK1 nucleic acid fragment comprises a nucleotide sequence set forth below (SEQ ID NO:3):

5′-
AACTGCATCA TTGGTAAAGG ACGCAGCTAC AAGGGAACAG
TATCTATCAC TTGACGTAGT AACCATTTCC TGCGTCGATG
TTCCCTTGTC ATAGATAGTG TAAGAGTGGC ATCAAATGTC
AGCCCTGGAG TTCCATGATA CCACACGAAC ATTCTCACCG
TAGTTTACAG TCGGGACCTC AAGGTACTAT GGTGTGCTTG
ACAGCTTTTT GCCTTCGAGC TATCGGGGTA AAGACCTACA
GGAAAACTAC TGTCGAAAAA CGGAAGCTCG ATAGCCCCAT
TTCTGGATGT CCTTTTGATG TGTCGAAATC CTCGAGGGGA
AGAAGGGGGA CCCTGGTGTT TCACAAGCAA ACAGCTTTAG
AGCTCCCCT TCTTCCCCCT GGGACCACAA AGTGTTCGTT
TCCAGAGGTA CGCTACGAAG TCTGTGACAT TCCTCAGTGT
TCAGAAGTTG AGGTCTCCAT GCGATGCTTC AGACACTGTA
AGGAGTCACA AGTCTTCAAC AATGCATGAC CTGC-3′
TTACGTACTG GACG

The isolated HGFK1 nucleic acid fragments can exist in any suitable form. For example, the isolated HGFK1 nucleic acid fragments can be a DNA, an RNA, or a mixture thereof. In addition, the isolated HGFK1 nucleic acid fragments can have any suitable modifications, e.g., can be a PNA.

A plasmid comprising the HGFK1 nucleic acid fragments is provided. Cells comprising the HGFK1 nucleic acid fragments are also provided. Any suitable cells can be used, e.g., bacterial cells, yeast cells, fungal cells, plant cells, insect cells and animal cells. Methods for producing HGFK1 are further provided, which methods comprise growing the cells comprising the HGFK1 nucleic acid fragments under conditions whereby the HGFK1 is expressed by the cells, and recovering the expressed HGFK1.

In another aspect, the invention provides a substantially purified HGFK1. Preferably, the substantially purified HGFK1 comprises the amino acid sequence set forth below:

(SEQ ID NO:1)

N′-CIIGKGRSYKGTVSITKSGIKCQPWSSMIPHEH-
SFLPSSYRGKDLQENYCRNPRGEEGGPWCFTSNPEVRYEVCDIPQC-C′
or

(SEQ ID NO:2)

N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

Also preferably, a conjugate is provided, which conjugate comprises: a) a HGFK1; and b) a facilitating agent linked to the HGFK1 directly or via a linker, wherein the agent facilitates: i) affinity isolation or purification of the conjugate; ii) attachment of the conjugate to a surface; or iii) detection of the conjugate. One exemplary conjugate is fusion protein between a HGFK1 and a facilitating protein or peptide.

The HGFK1 nucleic acids and proteins, their functional derivatives or fragments, or conjugates comprising a HGFK1 protein and a facilitating agent can be made by any suitable methods such as chemical synthesis, recombinant methods or a combination thereof. Preferably, the HGFK1 nucleic acids and proteins, their functional derivatives or fragments, or conjugates comprising a HGFK1 protein and a facilitating agent are made by recombinant methods (Current Protocols in Molecular Biology, Ausubel, et al. eds., John Wiley & Sons, Inc. (2000); and Sambrook, et al., Molecular Cloning: A Laboratory Manual, Cold Spring Harbor Laboratory press, (1989)).

In still another aspect, the invention provides a method for inhibiting pathological cell growth or angiogenesis, which method comprises administering to a subject to which such inhibition is needed or desirable, an effective amount of HGFK1, or a functional derivative or fragment thereof, or a nucleic acid encoding said HGFK1 or functional derivative or fragment thereof, or an agent that increases production and/or cell growth or angiogenesis inhibiting function of said HGFK1, thereby inhibiting said pathological cell growth or angiogenesis in said subject.

Any suitable subject can be treated by the present methods. Preferably, the subject to be treated is a mammal. More preferably, the subject to be treated is a human. The subject to be treated can have tumor, cancer or a disease or disorder associated with undesirable or pathological angiogenesis.

The HGFK1 protein or nucleic acid, or a functional derivative or fragment thereof, can be administered by any suitable methods. For example, the HGFK1 protein or nucleic acid, or a functional derivative or fragment thereof, can be administered by intracavernous injection, subcutaneous injection, intravenous injection, intramuscular injection, intradermal injection, or topical administration. Alternatively, the HGFK1 protein or nucleic acid, or a functional derivative or fragment thereof, can be administered via a gene therapy vector. Exemplary gene therapy vectors include an adenovirus associated vector, a retroviral vector, an adenovirus vector, and a lentivirus vector. Preferably, an adenovirus associated vector is used. Also alternatively, the HGFK1 protein or nucleic acid, or a functional derivative or fragment thereof, can be administered via a liposome.

Any suitable HGFK1 protein or nucleic acid, or a functional derivative or fragment thereof, can be used in the present methods. Preferably, the HGFK1 protein comprises the amino acid sequence set forth below is used:

(SEQ ID NO:1)

N′-CIIGKGRSYKGTVSITKSGIKCQPWSSMIPHEH-
SFLPSSYRGKDLQENYCRNPRGEEGGPWCFTSNPEVRYEVCDIPQC-C′
or

(SEQ ID NO:2)

N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

Also preferably, the HGFK1 nucleic acid comprises a nucleotide sequence that is hybridizable, under low, middle or high stringency, with a nucleotide sequence set forth below is used (SEQ ID NO:3):

5′-
AACTGCATCA TTGGTAAAGG ACGCAGCTAC AAGGGAACAG
TATCTATCAC TTGACGTAGT AACCATTTCC TGCGTCGATG
TTCCCTTGTC ATAGATAGTG TAAGAGTGGC ATCAAATGTC
AGCCCTGGAG TTCCATGATA CCACACGAAC ATTCTCACCG
TAGTTTACAG TCGGGACCTC AAGGTACTAT GGTGTGCTTG
ACAGCTTTTT GCCTTCGAGC TATCGGGGTA AAGACCTACA
GGAAAACTAC TGTCGAAAAA CGGAAGCTCG ATAGCCCCAT
TTCTGGATGT CCTTTTGATG TGTCGAAATC CTCGAGGGGA
AGAAGGGGGA CCCTGGTGTT TCACAAGCAA ACAGCTTTAG
GAGCTCCCCT TCTTCCCCCT GGGACCACAA AGTGTTCGTT
TCCAGAGGTA CGCTACGAAG TCTGTGACAT TCCTCAGTGT
TCAGAAGTTG AGGTCTCCAT GCGATGCTTC AGACACTGTA
AGGAGTCACA AGTCTTCAAC AATGCATGAC CTGC-3′
TTACGTACTG GACG

More preferably, the HGFK1 nucleic acid comprises a nucleotide sequence set forth below is used (SEQ ID NO:3):

5′-
AACTGCATCA TTGGTAAAGG ACGCAGCTAC AAGGGAACAG
TATCTATCAC TTGACGTAGT AACCATTTCC TGCGTCGATG
TTCCCTTGTC ATAGATAGTG TAAGAGTGGC ATCAAATGTC
AGCCCTGGAG TTCCATGATA CCACACGAAC ATTCTCACCG
TAGTTTACAG TCGGGACCTC AAGGTACTAT GGTGTGCTTG
ACAGCTTTTT GCCTTCGAGC TATCGGGGTA AAGACCTACA
GGAAAACTAC TGTCGAAAAA CGGAAGCTCG ATAGCCCCAT
TTCTGGATGT CCTTTTGATG TGTCGAAATC CTCGAGGGGA
AGAAGGGGGA CCCTGGTGTT TCACAAGCAA ACAGCTTTAG
GAGCTCCCCT TCTTCCCCCT GGGACCACAA AGTGTTCGTT
TCCAGAGGTA CGCTACGAAG TCTGTGACAT TCCTCAGTGT
TCAGAAGTTG AGGTCTCCAT GCGATGCTTC AGACACTGTA
AGGAGTCACA AGTCTTCAAC AATGCATGAC CTGC-3′
TTACGTACTG GACG

In yet another aspect, the invention provides a pharmaceutical composition for inhibiting pathological cell growth or angiogenesis, which pharmaceutical composition comprises an effective amount of HGFK1, or a functional derivative or fragment thereof, or a nucleic acid encoding said HGFK1 or functional derivative or fragment thereof, or an agent that increases production and/or cell growth or angiogenesis inhibiting function of said HGFK1. Preferably, The pharmaceutical composition further comprises a pharmaceutically acceptable carrier or excipient. A kit is also provided, which kit comprises the above pharmaceutical composition and an instruction for using said pharmaceutical composition in inhibiting pathological cell growth or angiogenesis.

In yet another aspect, the invention provides a combination for inhibiting pathological cell growth or angiogenesis, which combination comprises: a) an effective amount of an agent that inhibits pathological cell growth or angiogenesis; and b) an effective amount of HGFK1, or a functional derivative or fragment thereof, or a nucleic acid encoding said HGFK1 or functional derivative or fragment thereof, or an agent that increases production and/or cell growth or angiogenesis inhibiting function of said HGFK1, thereby inhibiting said pathological cell growth or angiogenesis in said subject. Preferably, the combination is in the form of a pharmaceutical composition. More preferably, the combination further comprises a pharmaceutically acceptable carrier or excipient. A method for inhibiting pathological cell growth or angiogenesis is also provided, which method comprises administering to a subject to which such inhibition is needed or desirable, an effective amount of the above combination, thereby inhibiting said pathological cell growth or angiogenesis in said subject. A kit is also provided, which kit comprises the above combination and an instruction for using said combination in inhibiting pathological cell growth or angiogenesis.

The formulation, dosage and route of administration of the above-described compositions, combinations, preferably in the form of pharmaceutical compositions, can be determined according to the methods known in the art (see e.g., Remington: The Science and Practice of Pharmacy, Alfonso R. Gennaro (Editor) Mack Publishing Company, April 1997; Therapeutic Peptides and Proteins: Formulation, Processing, and Delivery Systems, Banga, 1999; and Pharmaceutical Formulation Development of Peptides and Proteins, Hovgaard and Frkjr (Ed.), Taylor & Francis, Inc., 2000; Medical Applications of Liposomes., Lasic and Papahadjopoulos (Ed.), Elsevier Science, 1998; Textbook of Gene Therapy, Jain, Hogrefe & Huber Publishers, 1998; Adenoviruses: Basic Biology to Gene Therapy, Vol. 15, Seth, Landes Bioscience, 1999; Biopharmaceutical Drug Design and Development, Wu-Pong and Rojanasakul (Ed.), Humana Press, 1999; Therapeutic Angiogenesis: From Basic Science to the Clinic, Vol. 28, Dole et al. (Ed.), Springer-Verlag New York, 1999). The compositions, combinations or pharmaceutical compositions can be formulated for oral, rectal, topical, inhalational, buccal (e.g., sublingual), parenteral (e.g., subcutaneous, intramuscular, intradermal, or intravenous), transdermal administration or any other suitable route of administration. The most suitable route in any given case will depend on the nature and severity of the condition being treated and on the nature of the particular composition, combination or pharmaceutical composition which is being used.

### BRIEF DESCRIPTION OF THE SEVERAL VIEWS OF THE DRAWING(S)

FIG. 1. SDS-PAGE analysis of different fractions of protein. Samples were loaded onto a 15% gel followed by staining with Coomassie Blue. Lane 1, protein of total bacteria; Lane 2, soluble protein in E. coli; Lane 3, insoluble protein in E. coli; Lane 4, flow through; Lane 5, elution by washing buffer, pH 6.3; Lane 6, purified HGFK1; Lane 7, protein markers.

FIG. 2. HGFK1 and angiostatin inhibit the proliferation of BAE cells stimulated by bFGF. Values represent the mean of three determinations (±SE) by MTT assay.

FIG. 3. HGFK1 induces apoptosis in BAE cells. BAE cell monolayers were exposed overnight to HGFK1 (2 μg/ml) in 0.5% serum, and apoptosis was assayed by propidium iodide. Data were pooled from two experiments and results were expressed as the mean percentage (±SE) of cells with evidence of apoptosis. Column 1 control; column 2, cells treated with HGFK1 (2 μg/ml).

FIG. 4. Treatment of BAE cell with angiostatin or HGFK1 causes cell morphological changes. (a) control; (b) 5 ug/ml angiostatin added; (c) 2 ug/ml HGFK1 added.

FIG. 5. bFGF-induced down-regulation of caveolin-1 expression is time- and concentration-dependent. BAE cells were treated with 0-10 ng/ml bFGF for 24 h (concentration dependence, panel A) or with 10 ng/ml bFGF for a period of up to 24 h (time course, panel B). After the treatment, cells lysates were subjected to immunoblot analysis with isoform-specific antibody that detects caveolin-1. Each lane contains an equal amount of protein.

FIG. 6. Angiostatin and HGFK1 block the down-regulation of caveolin-1 expression in BAE cell. BAE cell were treated with or without 10 ng/ml bFGF for 24 h, in the presence or absence of angiostatin or HGFK1.

FIG. 7. Schematic diagram of a proposed mechanism, through which HGFK1 or angiostatin perform their activities. CA, SM, M and agt represent caveolin-1, signalling molecules, cell membrane and angiostatin (HGFK1), respectively.

### DETAILED DESCRIPTION OF THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, published patent applications and other publications and sequences from GenBank and other databases referred to herein are incorporated by reference in their entirety. If a definition set forth in this section is contrary to or otherwise inconsistent with a definition set forth in patents, published patent applications and other publications and sequences from GenBank and other data bases that are herein incorporated by reference, the definition set forth in this section prevails over the definition that is incorporated herein by reference.

As used herein, “a” or “an” means “at least one” or “one or more.”

As used herein, “Kringle domain 1 of human hepatocyte growth factor (HGFK1)” includes those variants with conservative amino acid substitutions that do not substantially alter their cell growth or angiogenesis inhibiting activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

As used herein, a “functional derivative or fragment of HGFK1 ” refers to a derivative or fragment of HGFK1 that still substantially retains its function as a pathological cell growth or angiogenesis inhibitor. Normally, the derivative or fragment retains at least 50% of its pathological cell growth or angiogenesis inhibiting activity. Preferably, the derivative or fragment retains at least 60%, 70%, 80%, 90%, 95%, 99% and 100% of its pathological cell growth or angiogenesis inhibiting activity.

As used herein, an “agent that enhances cell growth or angiogenesis inhibiting function of HGFK1” refers to a substance that increases potency of HGFK1's pathological cell growth or angiogenesis inhibiting activity, or a substance that increases sensitivity of a HGFK1's natural ligand in an pathological cell growth or angiogenesis inhibiting signaling pathway, or a substance that decreases potency of a HGFK1's antagonist.

As used herein, an “agent that enhances production of HGFK1” refers to a substance that increases transcription and/or translation of a HGFK1 gene, or a substance that increases post-translational modification and/or cellular trafficking of a HGFK1 precursor, or a substance that prolongs half-life of a HGFK1 protein.

As used herein, a “combination” refers to any association between two or among more items.

As used herein, a “composition” refers to a any mixture of two or more products or compounds. It may be a solution, a suspension, liquid, powder, a paste, aqueous, non-aqueous or any combination thereof.

As used herein, “nucleic acid” refers to any nucleic acid containing molecule including, but not limited to DNA, RNA or PNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein: “stringency of hybridization” in determining percentage mismatch is as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0. 1% SDS, 50° C. It is understood that equivalent stringencies may be achieved using alternative buffers, salts and temperatures.

For clarity of disclosure, and not by way of limitation, the detailed description of the invention is divided into the subsections that follow. The following example is included for illustrative purposes only and is not intended to limit the scope of the invention.

### B. EXAMPLES

### Example 1

### Kringle 1 of Human Hepatocyte Growth Factor Inhibits Bovine Aortic Endothelial Cell Proliferation Stimulated by Basic Fibroblast Growth Factor and Causes Cell Apoptosis

Materials and Methods

Reagents and materials. DMEM and Trypsin/EDTA were purchased from GIBCO BRL (Rockville, Md.). Fetal calf serum were from Hyclone (Logen Utah). Human fresh placenta was from Shanghai No. 1 women and children healthcare hospital. Angiostatin was prepared in our lab as described previously (9).

RT-PCR. Human fresh placenta was homogenized in liquid nitrogen. Total RNA was isolated using TRIZOL reagent (GIBCO BRL). Total RNA was used as the template for cDNA synthesis using Superscript™ RNase H⁻ transcriptase (GIBCO BRL) according to manufacturer's instructions. PCR was performed with Ex-Taq DNA polymarase (TaKaRa) according to manufacturer's instructions. The synthetic oligonucleotides were obtained from Shanghai Sangon Co. Ltd.(Shanghai China). The primers used were as follows: RT primer: 5′ GCAGGTCATGCATTCAAC 3′ (SEQ ID NO:4), primers used for amplifying cDNA encoding HGFK1, sense primer: 5′ GGAATTCCATATGAACTGCATCATTGGTAAAGGA 3′ (SEQ ID NO:5), antisense primer: 5′ ATCGAA GCTTATTAATGGTGGTGATGGTGGTGGCAGGTCATGCATTC (SEQ ID NO:6), a NdeI site was included in the sense primer, a HindIII site, a stop codon and six histidine codon were incorporated into antisense primer. PCR product of 290 bp was amplified with this primer sets. Reaction were incubated in PE480 thermal cycler (Perkin-Elmers, NJ) for 35 cycles: denaturation 30 s, 94° C.; annealing 30 s, 52° C.; extension 30 s, 72° C. PCR product was run on 1% agarose gel in TBE (10 mM Tris-Borate, 1 mM EDTA, pH 8.0), and visualized by ethidium bromide staining.

Gene construction and expression. The amplified cDNA fragment was ligated into the NdeI/HindIII sites of Escherichia coli expression vector pET24-a (Novagen), resulting in the expression plasmid pETHK1. pETHK1 was transformed into E. coli BL21 (DE3) and HGFK1 expression was induced by 1 mM IPTG. Cells were harvested by centrifugation for 30 min at 4000 g.

Purification and refolding of recombinant HGFK1. Cells were resuspended in 20 mM Tris-HCl, pH 8.0, 50 mM KCl, 0.5 mM EDTA, 5 mM DTE and lysozyme was added to the final concentration of 0.5 mg/ml. Cells were incubated at 4° C. for 30 min, then were disrupted by sonic homogenizer for 10 sec for six times with 30 sec interval each time. After centrifugation at 4° C., 12,000 g for 30 min, the pellet was collected and resuspended in 8 M urea, 0.1 M NaH₂PO₄, 10 mM Tris-HCl, pH 8.0. Centrifuged again as before, the supernatant was loaded on a Ni²⁺-nitrilotriacetic acid-agarose column (Qiagen). The recombinant protein was eluted from the column according to manufacturer's instructions. To achieve refolding, the purified protein were adjusted to pH 8.0 and DTT was added to the final concentration of 0.1 M. Following incubation at room temperature for 2 hours, the solution was added to refolding buffer (0.1 M Tris-HCl, pH 8.0, 0.5 M arginine, 5 mM EDTA, 1 mM GSSG, 5 mM GSH) with the ratio of 1:200(v/v). After 24 hours' incubation at room temperature, the renatured protein was dialyzed against distilled water for 24-48 hours and lyophilized.

Bovine aortic endothelial cell proliferation assay. Bovine aortic endothelial cell were isolated as previously described (10) and were maintained in DMEM supplemented with 10% heat-inactivated FCS and antibiotics. Monolayer of BAE cells growing in 60 mm dish were dispersed in 0.05% trypsin solution. Cells were resuspended with DMEM containing 10% FCS. Approximately 3000 cells in 200 ul were added in triplicate to each well of 96-well tissue culture plates and incubated at 37° C.(in 10% CO₂). Cells adhere to the plate in about 2-3 hours. The medium was replaced with 100 ul of fresh DMEM containing 2% FCS, and samples of HGFK1 or angiostatin were added to each well. After 30 minutes' incubation, another 100 ul DMEM containing 2% FCS and 10 ng/ml bFGF was added to each well. After 72 hours' incubation, 10 ul MTT (100 mg/ml) was added to each well and incubated for another 4 hours at 37° C., 10% CO₂. 180 ul medium was pipetted out from each well and 50 ul DMSO was added, vortex gently to dissolve the pellet. The absorbance of A_{570 nm}, which correlates to the number of cells, was measured with microplate reader (Model 450, Bio-Rad).

Flow cytometry apoptosis analysis by Propidium iodide (PI) assay. All the procedures were followed as previously reported (11). Briefly, BAE cells were maintained in DMEM supplemented with 10% FCS till to 60-70% confluence. The medium was changed with DMEM supplemented with 0.5% FCS containing 2 ug/ml HGFK1. An hour later, bFGF was added to the final concentration of 5 ng/ml and cells were cultured overnight at 37° C. in 10% CO₂. Cells were trypsinized and washed gently with PBS, and then were fixed with 70% ice-cold ethanol for 30 minutes. Cells were collected by centrifugation. 200 ul 1 mg/ml RNase was added and incubated at 37° C. for 30 minutes, then 400 ul PI(500 ug/ml) was added and incubated dark at 4° C. for 30 minutes. Cells were assessed by FACStar plus flow cytometer (Beckton-Dickinson) for apoptosis and the results were analyzed with CellQuest software.

Results

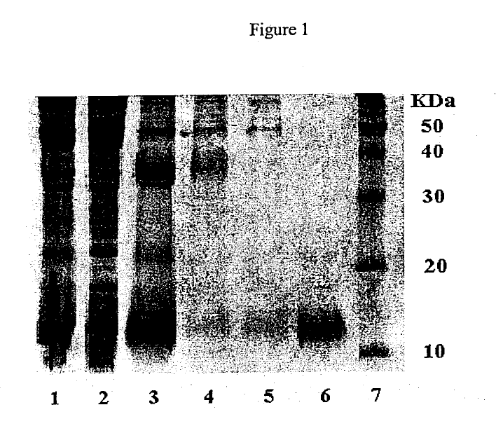

Purification and characterization of HGFK1. Recombinant protein including amino acids residues from 127-214 of HGF plus six histidines was expressed in E. coli. and purified to homogeneity using Ni²⁺-nitrilotriacetic acid-agarose column and was refolded in vitro (FIG. 1). Under reducing condition, HGFK1 migrated in PAGE with molecular mass of about 11 KD, corresponding to the predicted molecular mass.

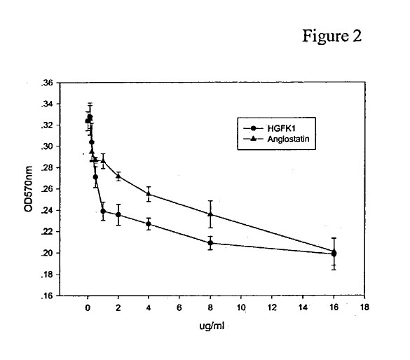

Inhibition of BAE cell proliferation with recombinant HGFK1 and human angiostatin. HGFK1 and angiostatin were assayed for their inhibitory activities on bovine aortic endothelial cell growth stimulated by bFGF. As shown in FIG. 2, both angiostatin and HGFK1 inhibited BAE cell proliferation in a dose-dependent fashion. The concentration of HGFK1 required to reach 50% inhibition (ED50) was about 0.7 ug/ml, while ED50 of angiostatin is 3 ug/ml, close to data previously reported (4). While both HGFK1 and angiostatin have no inhibitory activities on fibroblast cell line Balb/c3T3 and hepatoma cell line HepG2 (data not shown), which suggests that their inhibitory activity is specific to endothelial cell.

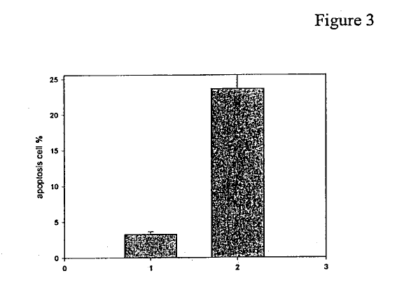

Cell apoptosis detection. As shown in FIG. 3, BAE cells were treated with 2 ug/ml HGFK1 overnight in 0.5% FCS, about 23% cells underwent apoptosis, compared with 3% of untreated cells.

Discussion

Kringle domains were found in many proteins, most of which are important molecules mediating coagulation and fibrinolysis or lipid transportation. Therefore, kringle modules were thought to function as recognition unit for protein and protein or protein and cell surface (1). However, in recent years, several proteins containing kringle module were found to inhibit specifically the proliferation of endothelial cell. Angiostatin, which is composed by the first four kringles of plasminogen was found to inhibit endothelial cell proliferation stimulated by bFGF (9) and except kringle 4, each kringle of plasminogen had more or less anti-angiogenesis activity (5). Lee et al. (6) reported that kringle 2 of prothrombin had inhibitory activity against BCE proliferation. Reports about apolipoprotein A were contradictory. Lou et al. (7) found that it displayed no such kind of activity despite that its kringle showed great sequence similarity with kringle 4 of plasminogen; while Trieu et al. (8) reported that recombinant apolipoprotein A with 18 kringle 4 repeats impaired angiogenesis in animal model experiment. These reports, combined together, implied that some kringle domains manifested anti-angiogenic activity. Amino acid sequence alignment of the kringle domains of plasminogen and HGFK1 showed that they displayed considerable similarity (40-50% identity, Table 1). We thus wondered whether HGFK1 was also an angiogenesis inhibitor. Our result in this paper demonstrated for the first time that HGFK1 is indeed an angiogenesis inhibitor.

TABLE 1
Amino acids sequence alignment of kringle domains of plasminogen and HGF

| | | |
|---|---|---|
| PK1 | CKTGNGKNYRGTMSKTKNGITCQKWSSTSPHRP-RFSPATHPSEGLEENYCRNPDNDPQG | (SEQ ID NO:7) |
| PK5 | CMFGNGKGYRGKRATTVTGTPCQDWAAQEPHRHSIFTPETNPRAGLEKNYCRNPDGDVGG | (SEQ ID NO:8) |
| PK4 | CYHGDGQSYRGTSSTTTTGKKCQSWSSMTPHRH-QKTPENYPNAGLTMNYCRNPDADK-G | (SEQ ID NO:9) |
| HK1 | CIIGKGRSYKGTVSITKSGIKCQPWSSMIPHEH-SFLPSSYRGKDLQENYCRNPRGEEGG | (SEQ ID NO:10) |
| PK2 | CMHCSGENYDGKISKTMSGLECQAWDSQSPHAH-GYIPSKFPNKNLKKNYCRNPDREL-R | (SEQ ID NO:11) |
| PK3 | CLKGTGENYRGNVAVTVSGHTCQHWSAQTPHTH-NRTPENFPCKNLDENYCREPDGKR-A | (SEQ ID NO:12) |
| | * :*..* *.:* .* ***: **.: *. :..* ****:*:. : | |
| PK1 | PWCYTTDPEKRYDYCDILEC | |
| PK5 | PWCYTTNPRKLYDYCDVPQC | |
| PK4 | PWCFTTDPSVRWEYCNLKKC | |
| HK1 | PWCFTSNPEVRYEVCDIPQC | |
| PK2 | PWCFTTDPNKRWELCDIPRC | |
| PK3 | PWCHTTNSQVRWEYCKIPSC | |
| | ***.*::. .:::.*::: * | |

The sequences of 5 kringle domains were aligned according to their conserved cysteines and amino acids clusters. PK represents the kringle of plasminogen, while HK represent the kringle of HGF. Conserved amino acids residues are marked with “*”;
Highly conserved residues are marked with “:”; Less conserved residues are marked with “.”.

In the examples above, the “mother” proteins display no inhibitory activity on angiogenesis and HGF is even a growth factor with angiogenic activity. Therefore, we proposed that in “mother” proteins the functional elements of kringle were shielded and could not interact with endothelial cell effectively. While after kringle domains were set free from “mother” molecules, they were more freely to access the binding sites on endothelial cell membrane and manifested anti-angiogenic function. This phenomenon might be regarded as the result of the diversity of protein function in molecule evolution.

Then, why different kringles inhibited the proliferation of endothelial cell in different degree? We contributed it to their subtle differences in amino acids sequence, as well as in their second structure. It has been reported that lysine-binding site in kringle was not related with inhibitory activity (4); the treatment of kringle structure by reductive reagent may compromise its inhibitory activity (4); Kringle 4 is distinctive among kringles of plasminogen for not having such inhibitory activity. Cao et al. (5) contributes it to two sets of consecutive lysine residues in its amino acids sequence. Therefore, much effort is needed to locate the exact amino acids clusters that count most.

As was seen in FIG. 2, HGFK1 was 3 times stronger than angiostatin in inhibiting the proliferation of endothelial cell. We believe that HGFK1 might share the same receptor with angiostatin on cell membrane with different Kd, which led to the different degree of their inhibitory activity. Moser et al reported that ATP synthase on the membrane of HUVEC cell line was the receptor of angiostatin (12). They suggested that angiostatin bound to ATP synthase and rendered endothelial cells more vulnerable to hypoxic challenge and eventual irreversible cell damage (12). However, direct evidence has not been reported as to whether binding of ATP synthase to angiostatin could block its enzymatic activity or not.

Angiostatin was reported to induce endothelial cell apoptosis (13). We here demonstrated that HGFK1 could induce BAE cell apoptosis, too. Little is known about their mechanism. Liu et al. reported that angiostatin treatment of ECV304, a well characterized human umbilical endothelial cell line, could block VEGF or bFGF induced down regulation of caveolin-1 (14). Caveolin-1 is the marker protein of caveolae, whose function was regarded to be transportation of materials from outside to inside cell membrane (15). ATP synthase was found in caveolae (16), which made us suspect that kringles in angiostatin and HGFK1 may inhibit cell proliferation stimulated by bFGF and induce cell apoptosis at least partly by disturbing material transportation through caveolae on cell membrane.

### Example 2

### Kringle 1 of Human Hepatocyte Growth Factor Differentially Regulates Caveolin-1 Expression in Bovine Aortic Endothelial Cell

Kringle domain is a kind of protein module, which usually consists of about 80 amino acids (1). Many proteins with this domain, for example, angiostatin and HGFK1, are reported to manifest anti-angiogenic activity (2)(5). We previously reported that HGFK1 inhibited specifically the proliferation of bovine aortic endothelial cell stimulated by bFGF, with ED₅₀ of about 0.7 ug/ml and caused BAE cell apoptosis (17). However, the exact mechanism by which it performs its activity is not clear yet.

Caveolin-1 is a membrane protein abundantly found in caveolae, the vesicular invaginations of plasma membrane (18). Its topology is unusual in that both its N-terminal and C-terminal domains face cytoplasm (15). Many important molecules, including G protein coupled receptors, receptor tyrosine kinase, are found to bind with caveolin in caveolae (15). Direct interaction of caveolin with signaling molecules leads to their inactivation (15). Liu et al reported that bFGF induced down-regulation of caveolin-1 in ECV304, a well characterized endothelial cell line, and that angiostatin could block this down-regulation (14). In this example, we demonstrated for the first time that HGFK1 could block the down-regulation of caveolin-1 induced by bFGF in BAE cell line. Therefore, all the proteins with kringle domain might display their inhibitory activity via same mechanism in endothelial cells.

Materials and Methods

Reagents and materials. DMEM and Trypsin/EDTA were purchased from GIBCO BRL (Rockville, Md.). Fetal calf serum were from Hyclone (Logen Utah). Polyclonal anti-caveolin-1 IgG was from Santa Cruz Biotechnologies, Inc. (Santa Cruz, Calif.). Angiostatin and HGFK1 were prepared in our lab as described previously (17)(9).

Cell culture and treatment with angiostatin and HGFK1. Bovine aortic endothelial cells were isolated as previously described (10) and were seeded at a density of ˜1.0×10⁴ cells/ml in 24-well plates, maintained in DMEM supplemented with 10% heat-inactivated FCS and antibiotics. After incubation in normal growth medium overnight, the medium was replaced by DMEM containing 5% fetal calf serum with or without bFGF, angiostatin or HGFK1 at different concentrations according to the purpose of the assay.

Protein Analysis. Expression of caveolin-1 was examined by Western Blot analysis. Cells were solubilized with sample buffer containing 0.125 M Tris-HCl (pH 6.8), 5% (w/v) SDS, 2.5%(v/v) β-mercaptoethanol, 5% glycerol in double distilled water. After boiling for 4 min, proteins were separated by SDS-polyacrylamide gel electrophoresis (5-15% gradient gel), transferred to nitrocelloses, and subjected to Western blot analysis using enhanced chemilunescence. Prior to loading, the protein concentration of the samples was measured with the bicinchoninic acid method using bovine serum albumin as a standard.

Results

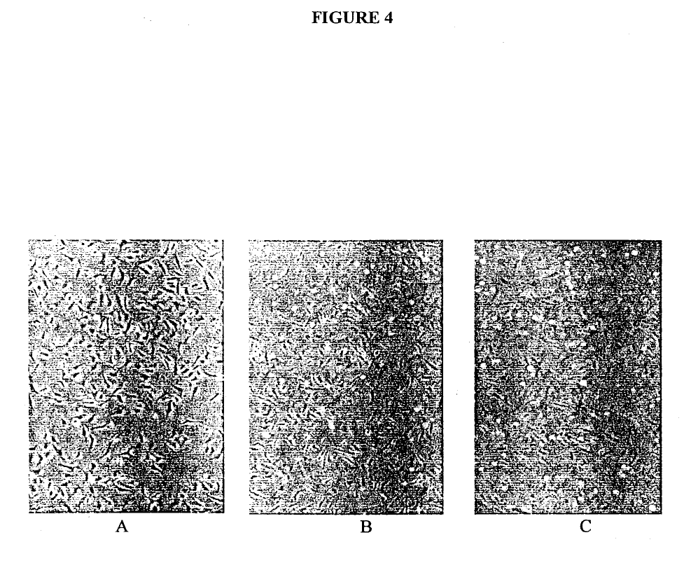

Angiostatin and HGFK1 cause morphological changes in BAE cell. Normally, bovine aortic endothelial cells adhere tightly to the cell dish as shown in FIG. 4(a). However, after exposure under 5 ug/ml angiostatin or 2 ug/ml HGFK1 for 24 hours, distinct morphological changes were observed. As were shown in FIGS. 4(b) and (c), some of the cells retracted from the cell dish and turned round morphologically under microscope.

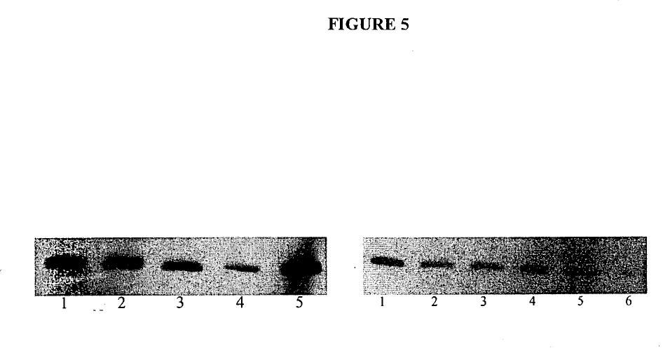

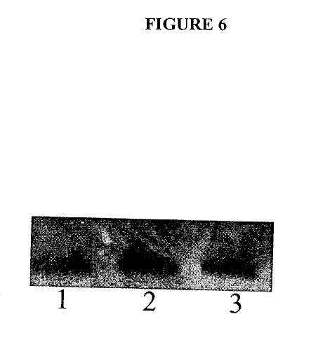

HGFK1 differentially regulates caveolin-1 expression in BAE cell. bFGF was reported to down-regulate the expression of caveolin-1 protein in ECV 304 cell line. Here we demonstrated that bFGF had the same effect on BAE cell line. We determined the time and concentration dependence of the effects of bFGF. As was shown in FIG. 5(a), bFGF induced the down-regulation of caveolin-1 at a minimal concentration of 3 ng/ml, and the effect became maximal at 10 ng/ml. bFGF-induced down-regulation of caveolin-1 appeared after 3 h of treatment, exerting its maximal effects at 24 h. (FIG. 5b). The down-regulation of caveolin-l by bFGF was blocked by 5 ug/ml angiostatin or 2 ug/ml HGFK1 (FIG. 6).

Discussion

Seven years have passed since the first publication of angiostatin and the related kringle domains. After that, a large number of literatures have been published to help advance their practicality in clinical research. However, few proceeding is accomplished as to the exact mechanism, by which they perform their activities. In this example, we try to help throw some light in this area.

Up till now, there is only one published explanation as to how angiostatin inhibits the metastasis of endothelial cell: kinetic analysis demonstrated that angiostatin functions as a non-competitive inhibitor of extracellular-matrix-enhanced, tPA-catalysed plasminogen activator (19); While there are two kinds of mechanisms to interpret how angiostatin and molecules alike inhibit the proliferation of endothelial cell. First, Moser et al reported that angiostatin bound to ATP synthase on HUVEC membrane. They speculated that the plasma membrane associated ATP synthase may produce extracellular ATP, which can diffuse back into the cell, providing an additional, albeit limited, ATP source. Binding of angiostatin with ATP synthase disrupted the production of ATP and rendered endothelial cell more vulnerable to hypoxic challenge and eventual irreversible damage (12). Second, Liu et al demonstrated that angiostatin could block the down-regulation of caveolin-1 in ECV304 cell line and thus affect the MAPK signaling pathway (14). In this example, we demonstrated for the first time that HGFK1 and angiostatin could block the down-regulation of caveolin-1 in BAE cell line. Therefore, we assume that all the proteins with kringle domain might perform their activities through the same mechanism in endothelial cells.

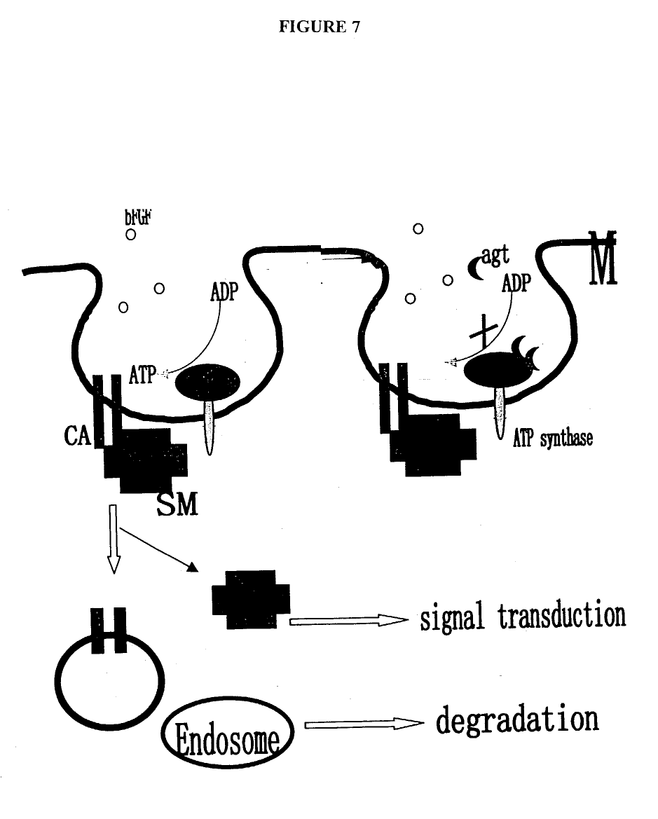

It is worthwhile to mention that ATP synthase is found in caveolae (16), therefore, it is very interesting to propose, as is shown in FIG. 7, that, besides the stimulation with bFGF, the dissociation of caveolin-1 and signaling molecules in caveolae might need energy, which could be provided by ATP synthase nearby in caveolae. After the dissociation, caveolin-1 is degraded via endosome pathway, so, once the cells are stimulated with bFGF, caveolin-1 is down regulated. However, if HGFK1 or angiostatin is added to the cell culture medium, it binds to ATP synthase and blocks the production of ATP, which makes the dissociation of caveolin-1 and the signaling molecules impossible. In that case, cells fail to proliferate and the caveolin-1 is not down regulated, which is compatible with our result.

### REFERENCES

1. Castellino, F. J. and McCance, S. The kringle domains of human plasminogen. From Plasminogen-related growth factors. (Bock, G. R. eds) John Wiley & sons, 1997.

2. Hepatocyte growth factor and c-met. (Goldburg, I. D. eds), EXS, Birkhaeuser, 1993.

3. O'Reilly, M. S., Holmgren, L., Shing Y., Catherine, Chen, Rosenthal, R. A., Moses, M., Lane, W. S., Cao, Y H., Sage, E. H. and Folkman, J. (1994) Cell 79,315-328.

4. Cao Y. H., Richard, W. J., Davidson, D., Schaller, J., Marti, D., Sohndel, S., McCane, S. G., O'Reilly, M S., Llinas, M and Folkman, J.(1996) J. Biol. Chem. 271(46), 29461-29467.

5. Cao Y. H., Andrew C. and Seong S. A.(1997) J. Biol. Chem. 272(36), 22924-22928.

6. Lee T H., Tai Y R. and Soung S K.(1998) J. Biol. Chem. 273(44), 28805-28812.

7. Lou X. J., Kwan H. H., Prionas S. D. (1998) Exp. Mol. Pathol. 65(2) 53-63.

8. Trieu, V. N. and Uckun, F. M. (1999) Biochem. Biophy. Res. Commun. 257, 714-718.

9. Sim B. K. L., O'Reilly, M S., Liang H., fortier, A. H., He, W X., Madsen, J. W., Lapcevich, R. and Nacy, C. A. (1997) Cancer Res. 57, 1329-1334.

10. Pepper M. S., Montesano R, El Aoumari A, Gros D, Orci L, Meda P. (1992) Am. J Physiol 262: C1246.

11. Lucas, L., Holmgren, I., Garcia, B., Jimenez, S. J., Mandriota, F., Borlat, B. K. L., Sim, Z., Grau, G. E., Shing, Y., Scoff G. A., Bouck, N. and Pepper, M. S. (1998) Blood, 92 (12),4730-4741.

12. Moser, T. L., Stack, M. S., Asplin, I., Enghild, J. J., Hojrup, P., Everitt, L., Hubchak, S., Schnaper, H. W. and Pizzo, S. V. (1998) Proc. Natl. Acad. Sci. USA 96,2811-2816.

13. Claesson-Welsh, L., Welsh, M., Ito, N., Anand-Apte, B., soker, S., Zetter, B., O'reilly, M S. and Folkman, J. (1998) Proc. Natl. Acad. Sci. USA 95, 5579-5583.

14. Liu, J., Razani, B., Tang S. Q., Terman, B. I., Ware, A. and Lisanti, M. P.(1999) J. Biol. Chem. 274(22), 15781-15785.

15. Okamoto, T., Schlegel, A., Scherer, P. E. and Lisandi, M. P. (1998) J. Biol. Chem. 273(10), 5419-5422.

16. Zetter, B. R. (1998) Annu. Rev. Med. 49,407-424.

17. Xin L, Xu R, Zhang Q, Li Z. P., Gan R. B. (2000) Biochem. Biophy. Res. Communn. 277(1) 186-190.

18. Sargiacomo M, Scherer P. E., Tang Z, Kubler E, Song K. S., Sanders M. C., Lisanti M. P. (1995) Proc. Natl. Acad. Sci. U.S.A. 92:9407-9411.

19. Stack M. S., Gately S, Bafetti L. M., Enghild J. J., Soff G. A. (1999) Biochem. J. 340:77-84

The above examples are included for illustrative purposes only and is not intended to limit the scope of the invention. Since modifications will be apparent to those of skill in this art, it is intended that this invention be limited only by the scope of the appended claims.

1. An isolated nucleic acid fragment, comprising a sequence of nucleotides encoding a Kringle domain 1 of human hepatocyte growth factor (HGFK1).

2. The isolated nucleic acid fragment of claim 1, which encodes the amino acid sequence set forth below:
(SEQ ID NO:1)
N′-CIIGKGRSYKGTVSITKSGIKCQPWSSMIPHEH-
SFLPSSYRGKDLQENYCRNPRGEEGGPWCFTSNPEVRYEVCDIPQC-C′
or
(SEQ ID NO:2)
N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

3. The isolated nucleic acid fragment of claim 1, which is hybridizable with a nucleotide sequence set forth below (SEQ ID NO:3):
5′-
AACTGCATCA TTGGTAAAGG ACGCAGCTAC AAGGGAACAG
TATCTATCAC TTGACGTAGT AACCATTTCC TGCGTCGATG
TTCCCTTGTC ATAGATAGTG TAAGAGTGGC ATCAAATGTC
AGCCCTGGAG TTCCATGATA CCACACGAAC ATTCTCACCG
TAGTTTACAG TCGGGACCTC AAGGTACTAT GGTGTGCTTG
ACAGCTTTTT GCCTTCGAGC TATCGGGGTA AAGACCTACA
GGAAAACTAC TGTCGAAAAA CGGAAGCTCG ATAGCCCCAT
TTCTGGATGT CCTTTTGATG TGTCGAAATC CTCGAGGGGA
AGAAGGGGGA CCCTGGTGTT TCACAAGCAA ACAGCTTTAG
GAGCTCCCCT TCTTCCCCCT GGGACCACAA AGTGTTCGTT
TCCAGAGGTA CGCTACGAAG TCTGTGACAT TCCTCAGTGT
TCAGAAGTTG AGGTCTCCAT GCGATGCTTC AGACACTGTA
AGGAGTCACA AGTCTTCAAC AATGCATGAC CTGC-3′
TTACGTACTG GACG

4. The isolated nucleic acid fragment of claim 1, which comprises a nucleotide sequence set forth below (SEQ ID NO:3):
5′-
AACTGCATCA TTGGTAAAGG ACGCAGCTAC AAGGGAACAG
TTGACGTAGT AACCATTTCC TGCGTCGATG TTCCCTTGTC
TATCTATCAC TAAGAGTGGC ATCAAATGTC AGCCCTGGAG
ATAGATAGTG ATTCTCACCG TAGTTTACAG TCGGGACCTC
TTCCATGATA CCACACGAAC ACAGCTTTTT GCCTTCGAGC
AAGGTACTAT GGTGTGCTTG TGTCGAAAAA CGGAAGCTCG
TATCGGGGTA AAGACCTACA GGAAAACTAC TGTCGAAATC
ATAGCCCCAT TTCTGGATGT CCTTTTGATG ACAGCTTTAG
CTCGAGGGGA AGAAGGGGGA CCCTGGTGTT TCACAAGCAA
GAGCTCCCCT TCTTCCCCCT GGGACCACAA AGTGTTCGTT
TCCAGAGGTA CGCTACGAAG TCTGTGACAT TCCTCAGTGT
AGGTCTCCAT GCGATGCTTC AGACACTGTA AGGAGTCACA
TCAGAAGTTG AATGCATGAC CTGC -3′
AGTCTTCAAC TTACGTACTG GACG

5. The isolated nucleic acid fragment of claim 1, which is a DNA.

6. The isolated nucleic acid fragment of claim 1, which is an RNA.

7. A plasmid, comprising the nucleic acid fragment of claim 1.

8. A cell, comprising the plasmid of claim 7.

9. The cell of claim 8 selected from the group consisting of a bacterial cell, a yeast cell, a fungal cell, a plant cell, an insect cell and an animal cell.

10. A method for producing HGFK1, comprising growing the cell of claim 8 under conditions whereby the HGFK1 is expressed by the cell, and recovering the expressed HGFK1.

11. A substantially purified HGFK1.

12. The substantially purified HGFK1 of claim 11, which comprises the amino acid sequence set forth below:
(SEQ ID NO:1)
N′-CIIGKGRSYKGTVSITKSGIKCQPWSSMIPHEH-
SFLPSSYRGKDLQENYCRNPRGEEGGPWCFTSNPEVRYEVCDIPQC-C′
or
(SEQ ID NO:2)
N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

13. A conjugate, comprising:
a) a HGFK1; and
b) a facilitating agent linked to the HGFK1 directly or via a linker, wherein the agent facilitates:
i) affinity isolation or purification of the conjugate;
ii) attachment of the conjugate to a surface; or
iii) detection of the conjugate.

14. A method for inhibiting pathological cell growth or angiogenesis, which method comprises administering to a subject to which such inhibition is needed or desirable, an effective amount of HGFK1, or a functional derivative or fragment thereof, or a nucleic acid encoding said HGFK1 or functional derivative or fragment thereof, or an agent that increases production and/or cell growth or angiogenesis inhibiting function of said HGFK1, thereby inhibiting said pathological cell growth or angiogenesis in said subject.

15. The method of claim 14, wherein the subject is a mammal.

16. The method of claim 15, wherein the mammal is a human.

17. The method of claim 14, wherein the HGFK1 protein or nucleic acid, or a functional derivative, or fragment thereof, or an agent that increases production and/or cell growth or angiogenesis inhibiting function of the HGFK1, is administered by intracavernous injection, subcutaneous injection, intravenous injection, intramuscular injection, intradermal injection, or topical administration.

18. The method of claim 14, wherein the HGFK1 protein or nucleic acid, or a functional derivative or fragment thereof, is administered via a gene therapy vector.

19. The method of claim 18, wherein the gene therapy vector is selected from the group consisting of an adenovirus associated vector, a retroviral vector, an adenovirus vector, and a lentivirus vector.

20. The method of claim 19, wherein the gene therapy vector is an adenovirus associated vector.

21. The method of claim 14, wherein the HGFK1 protein or nucleic acid, or a functional derivative or fragment thereof, or an agent that increases production and/or cell growth or angiogenesis inhibiting function of the HGFK1, is administered via a liposome.

22. The method of claim 14, wherein the subject has tumor or cancer.

23. The method of claim 14, wherein the subject has a disease or disorder associated with undesirable or pathological angiogenesis.

24. The method of claim 14, wherein the HGFK1 protein comprises the amino acid sequence set forth below:
(SEQ ID NO:1)
N′-CIIGKGRSYKGTVSITKSGIKCQPWSSMIPHEH-
SFLPSSYRGKDLQENYCRNPRGEEGGPWCFTSNPEVRYEVCDIPQC-C′
or
(SEQ ID NO:2)
N′-
CIIGKGRSYKGTVSITKSGIKCQPWSSMIPHEHSFLPSSYRGKDLQENY
CRNPRGEEGGPWCFTSNPEVRYEVCDIPQCSEVECMT-C′

25. The method of claim 14, wherein the HGFK1 nucleic acid comprises a nucleotide sequence that is hybridizable with a nucleotide sequence set forth below (SEQ ID NO:3):
5′-
AACTGCATCA TTGGTAAAGG ACGCAGCTAC AAGGGAACAG
TTGACGTAGT AACCATTTCC TGCGTCGATG TTCCCTTGTC
TATCTATCAC TAAGAGTGGC ATCAAATGTC AGCCCTGGAG
ATAGATAGTG ATTCTCACCG TAGTTTACAG TCGGGACCTC
TTCCATGATA CCACACGAAC ACAGCTTTTT GCCTTCGAGC
AAGGTACTAT GGTGTGCTTG TGTCGAAAAA CGGAAGCTCG
TATCGGGGTA AAGACCTACA GGAAAACTAC TGTCGAAATC
ATAGCCCCAT TTCTGGATGT CCTTTTGATG ACAGCTTTAG
CTCGAGGGGA AGAAGGGGGA CCCTGGTGTT TCACAAGCAA
GAGCTCCCCT TCTTCCCCCT GGGACCACAA AGTGTTCGTT
TCCAGAGGTA CGCTACGAAG TCTGTGACAT TCCTCAGTGT
AGGTCTCCAT GCGATGCTTC AGACACTGTA AGGAGTCACA
TCAGAAGTTG AATGCATGAC CTGC -3′
AGTCTTCAAC TTACGTACTG GACG

26. The method of claim 14, wherein the HGFK1 nucleic acid comprises a nucleotide sequence set forth below (SEQ ID NO:3):
5′-
AACTGCATCA TTGGTAAAGG ACGCAGCTAC AAGGGAACAG
TTGACGTAGT AACCATTTCC TGCGTCGATG TTCCCTTGTC
TATCTATCAC TAAGAGTGGC ATCAAATGTC AGCCCTGGAG
ATAGATAGTG ATTCTCACCG TAGTTTACAG TCGGGACCTC
TTCCATGATA CCACACGAAC ACAGCTTTTT GCCTTCGAGC
AAGGTACTAT GGTGTGCTTG TGTCGAAAAA CGGAAGCTCG
TATCGGGGTA AAGACCTACA GGAAAACTAC TGTCGAAATC
ATAGCCCCAT TTCTGGATGT CCTTTTGATG ACAGCTTTAG
CTCGAGGGGA AGAAGGGGGA CCCTGGTGTT TCACAAGCAA
GAGCTCCCCT TCTTCCCCCT GGGACCACAA AGTGTTCGTT
TCCAGAGGTA CGCTACGAAG TCTGTGACAT TCCTCAGTGT
AGGTCTCCAT GCGATGCTTC AGACACTGTA AGGAGTCACA
TCAGAAGTTG AATGCATGAC CTGC -3′
AGTCTTCAAC TTACGTACTG GACG

27. A pharmaceutical composition for inhibiting pathological cell growth or angiogenesis, which pharmaceutical composition comprises an effective amount of HGFK1, or a functional derivative or fragment thereof, or a nucleic acid encoding said HGFK1 or functional derivative or fragment thereof.

28. The pharmaceutical composition of claim 27, which further comprises a pharmaceutically acceptable carrier or excipient.

29. A combination for inhibiting pathological cell growth or angiogenesis, which combination comprises:
a) an effective amount of an agent that inhibits pathological cell growth or angiogenesis; and
b) an effective amount of HGFK1, or a functional derivative or fragment thereof, or a nucleic acid encoding said HGFK1 or functional derivative or fragment thereof, or an agent that increases production and/or cell growth or angiogenesis inhibiting function of said HGFK1, thereby inhibiting said pathological cell growth or angiogenesis in said subject.

30. The combination of claim 29, wherein the combination is in the form of a pharmaceutical composition.

31. The combination of claim 30, which further comprises a pharmaceutically acceptable carrier or excipient.

32. A method for inhibiting pathological cell growth or angiogenesis, which method comprises administering to a subject to which such inhibition is needed or desirable, an effective amount of the combination of claim 29, thereby inhibiting said pathological cell growth or angiogenesis in said subject.

33. A kit, which kit comprises the pharmaceutical composition of claim 27 and an instruction for using said pharmaceutical composition in inhibiting pathological cell growth or angiogenesis.

34. A kit, which kit comprises the combination of claim 29 and an instruction for using said combination in inhibiting pathological cell growth or angiogenesis.
